(19) United States
(12) Patent Application Publication
(10) Pub. No.: US 2026/0110679 A1
(43) Pub. Date: Apr. 23, 2026

(54) OPTICAL SENSOR ASSEMBLY FOR A SEED-PLANTING IMPLEMENT

(71) Applicant: CNH Industrial America LLC, New Holland, PA (US)

(72) Inventor: Michael Christopher Conboy, Chicago, IL (US)

(21) Appl. No.: 18/924,775

(22) Filed: Oct. 23, 2024

(51) Int. Cl. G01N 33/24; A01C 5/06; G01N 21/01

(52) U.S. Cl. CPC G01N 33/245; A01C 5/064; G01N 2021/0106

(57) ABSTRACT

An optical sensor assembly for a seed-planting implement includes a furrow firmer configured to shape a furrow being formed in soil by the seed-planting implement, with the furrow firmer extending in a vertical direction from a top end to a bottom end. The furrow firmer defines a cavity at the bottom end. Furthermore, the optical sensor assembly includes a non-electrically conductive housing positioned within the cavity and an optical sensor positioned within the cavity and coupled to the non-electrically conductive housing. Additionally, the optical sensor assembly includes a lens through which the optical sensor views the soil defining the furrow.

### FIELD OF THE INVENTION

The present disclosure generally relates to seed-planting implements and, more particularly, to an optical sensor assembly for a seed-planting implement.

### BACKGROUND OF THE INVENTION

Modern farming practices strive to increase yields of agricultural fields. In this respect, seed-planting implements are towed behind a tractor or other work vehicle to disperse seed throughout a field. For example, seed-planting implements typically include one or more furrow-forming tools or openers that excavate a furrow or trench in the soil. One or more dispensing devices of the seed-planting implements may, in turn, deposit the seeds into the furrow(s). After deposition of the seeds, a furrow-closing assembly may close the furrow in the soil, such as by pushing the excavated soil into the furrow.

The organic material content of the soil within the field is an important parameter when controlling the operation of the seed-planting implement. In this respect, optical sensors and sensor assemblies for seed-planting implements have been developed. While such sensors and sensor assemblies work well, further improvements are needed.

Accordingly, an improved optical sensor assembly for a seed-planting implement would be welcomed in the technology.

### SUMMARY OF THE INVENTION

Aspects and advantages of the technology will be set forth in part in the following description, or may be obvious from the description, or may be learned through practice of the technology.

In one aspect, the present subject matter is directed to an optical sensor assembly for a seed-planting implement. The optical sensor assembly includes a furrow firmer configured to shape a furrow formed in soil by the seed-planting implement, with the furrow firmer extending in a vertical direction from a top end to a bottom end. The furrow firmer defines a cavity at the bottom end. Furthermore, the optical sensor assembly includes a non-electrically conductive housing positioned within the cavity and an optical sensor positioned within the cavity and coupled to the non-electrically conductive housing. Additionally, the optical sensor assembly includes a lens through which the optical sensor views the soil defining the furrow.

In another aspect, the present subject matter is directed to a row unit for a seed-planting implement. The row unit includes a row unit frame and a disk opener rotatably coupled to the row unit frame, with the disk opener configured to form a furrow within the soil of a field as the seed-planting implement travels across the field. Moreover, the row unit includes a furrow firmer coupled to the row unit frame, with the furrow firmer configured to shape a furrow being formed in soil by the seed-planting implement, with the furrow firmer extending in a vertical direction from a top end to a bottom end. The furrow firmer defines a cavity at the bottom end. Furthermore, the optical sensor assembly includes a non-electrically conductive housing positioned within the cavity and an optical sensor positioned within the cavity and coupled to the non-electrically conductive housing. Additionally, the optical sensor assembly includes a lens through which the optical sensor views the soil defining the furrow.

In a further aspect, the present subject matter is directed to seed-planting implement including a toolbar and a plurality of row units supported on the toolbar. At least one row unit of the plurality of row units includes a furrow firmer configured to shape a furrow being formed in soil by the seed-planting implement, with the furrow firmer extending in a vertical direction from a top end to a bottom end. The furrow firmer defines a cavity at the bottom end. Furthermore, the at least one row unit of the plurality of row units includes a non-electrically conductive housing positioned within the cavity and an optical sensor positioned within the cavity and coupled to the non-electrically conductive housing. Additionally, the at least one row unit of the plurality of row units includes a lens through which the optical sensor views the soil defining the furrow.

These and other features, aspects, and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which refers to the appended figures, in which:

FIG. 1 illustrates a perspective view of one embodiment of a seed-planting implement in accordance with aspects of the present subject matter;

FIG. 2 illustrates a side view of one embodiment of a row unit of a seed-planting implement in accordance with aspects of the present subject matter; and

FIG. 3 illustrates a cross-sectional view of one embodiment of an optical sensor assembly in accordance with aspects of the present subject matter.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

As used herein, the term “and/or,” when used in a list of two or more items, means that any one of the listed items can be employed by itself, or any combination of two or more of the listed items can be employed. For example, if a composition or assembly is described as containing components A, B, and/or C, the composition or assembly can contain A alone; B alone; C alone; A and B in combination; A and C in combination; B and C in combination; or A, B, and C in combination.

In general, the present subject matter is directed to an optical sensor assembly for a seed-planting implement. As will be described below, the seed-planting implement includes a furrow firmer configured to shape the furrow being formed in the soil by the row unit. In this respect, the furrow firmer extends in a vertical direction from a top end to a bottom end, with the furrow firmer defining a cavity at the bottom end.

Additionally, the optical sensor assembly includes a non-electrically conductive housing, an optical sensor, and a lens. Specifically, in several embodiments, the non-electrically conductive housing is positioned within the cavity. For example, the non-electrically conductive housing may be coupled to the furrow firmer via one or more fasteners. Moreover, the optical sensor is positioned within the cavity and coupled to the non-electrically conductive housing. In this respect, the optical sensor views the soil defining the furrow through the lens. The lens, in turn, may be coupled (e.g., adhesively coupled) to the non-electrically conductive housing. Thus, during operation of the seed-planting implement, the optical sensor can generate data indicative of the organic matter content of the soil forming the bottom surface of the furrow.

The disclosed optical sensor improves the operation of the seed-planting implement. More specifically, as described above, the optical sensor of the disclosed optical sensor assembly can generate data indicative of the organic matter content present in the soil forming the bottom surface of the furrow. Moreover, the disclosed optical sensor assembly is positioned within the bottom end of the furrow firmer such that the optical sensor can generate such data while not negatively impacting the furrow closing operation unlike conventional optical sensors that bolt onto the seed-planting implement behind the furrow firmers. This, in turn, improves the agricultural performance of the field.

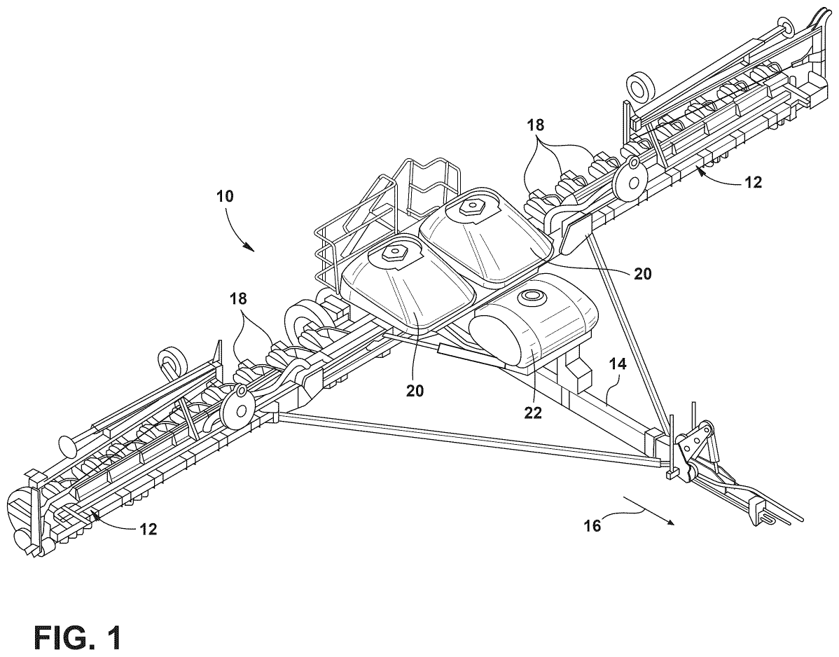

Referring now to drawings, FIG. 1 illustrates a perspective view of one embodiment of a seed-planting implement 10. In the illustrated embodiment, the seed-planting implement 10 is configured as a planter. However, in alternative embodiments, the seed-planting implement 10 may be configured as a seeder, a strip-tiller, a side-dresser, or any other suitable agricultural implement that deposits seeds into a field.

As shown in FIG. 1, the seed-planting implement 10 may include a laterally extending toolbar 12. More specifically, the toolbar 12 is connected at its middle to a forwardly extending tow bar 14 to allow the seed-planting implement 10 to be towed by a work vehicle (not shown), such as an agricultural tractor, in a direction of travel 16. In this respect, the toolbar 12 is generally configured to support a plurality of seed planting units or row units 18. Each row unit 18, in turn, is configured to deposit seeds at a desired depth beneath the soil surface and with a desired seed spacing as the seed-planting implement 10 travels across the field in the direction of travel 16, thereby establishing rows of planted seeds. In some embodiments, the bulk of the seeds to be planted may be stored in one or more hoppers or seed tanks 20. Thus, as seeds are planted by the row units 18, a pneumatic distribution system may distribute additional seeds from the seed tanks 20 to the individual row units 18. Additionally, one or more fluid tanks 22 may store agricultural fluids, such as insecticides, herbicides, fungicides, fertilizers, and/or the like. These fluids, in turn, may be supplied to the row units 18 for spraying onto the seeds during planting.

For purposes of illustration, only a portion of the row units 18 of the seed-planting implement 10 has been shown in FIG. 1. In general, the seed-planting implement 10 may include any number of row units 18, such as 6, 8, 12, 16, 24, 32, or 36 row units. In addition, the lateral spacing between row units 18 may be selected based on the type of crop being planted. For example, the row units 18 may be spaced approximately 30 inches from one another for planting corn, and approximately 15 inches from one another for planting soybeans.

The configuration of the seed-planting implement 10 described above and shown in FIG. 1 is provided only to place the present subject matter in an exemplary field of use. Thus, the present subject matter may be readily adaptable to any manner of seed-planting implement configuration.

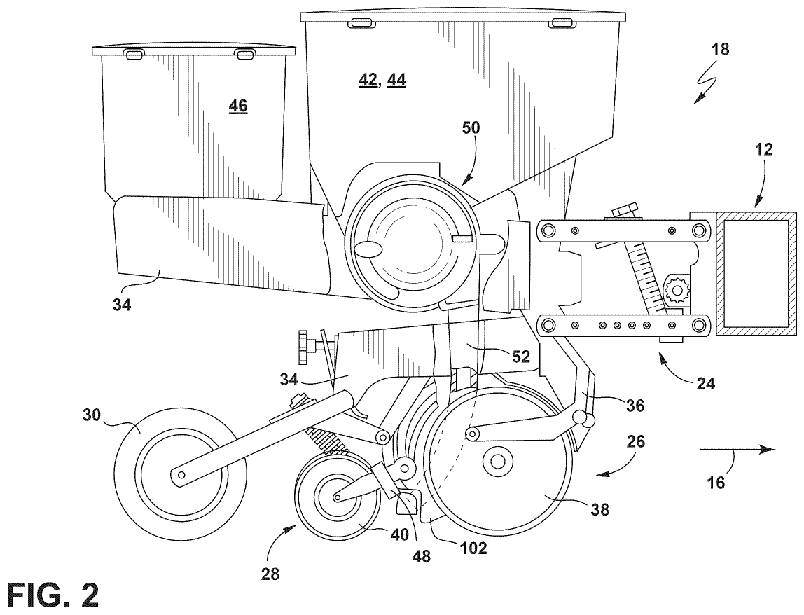

FIG. 2 illustrates a side view of one embodiment of a row unit 18. As shown, the row unit 18 includes a linkage assembly 24 configured to mount the row unit 18 to the toolbar 12 of the seed-planting implement 10. Furthermore, the row unit 18 also includes a row unit frame 34. In this respect, the row unit 18 may include a furrow opening assembly 26, a furrow closing assembly 28, and a press wheel 30 supported on or otherwise coupled row unit frame 34. In general, the furrow opening assembly 26 may include a gauge wheel (not shown) operatively coupled to the row unit frame 34 via a support arm 36. Additionally, the opening assembly 26 may also include one or more disk openers 38 rotatably coupled to the row unit frame 34. Moreover, the row unit 18 includes a furrow firmer 102 coupled to the row unit frame 34. The gauge wheel is not shown in FIG. 2 to better illustrate the disk opener(s) 38 and furrow firmer 102. The disk opener(s) 38 is configured to form or otherwise excavate a furrow or trench within the soil of a field as the seed-planting implement 10 (FIG. 1) travels across the field in the direction of travel 16. In this respect, the furrow firmer 102 is configured to shape the furrow formed in soil by the disk opener(s) 38 and firm the walls of such firm to prevent premature collapse of the furrow. In addition, the gauge wheel is configured to roll along or otherwise engage the surface of the field such that the position of the gauge wheel relative to the row unit frame 34 sets the depth of the furrow being excavated. Furthermore, as shown, the furrow closing assembly 28 may include a closing disk(s) 40 configured to close or collapse the furrow after seeds have been deposited therein. Thereafter, the press wheel 30 may roll over the closed furrow to firm the soil over the seed and promote favorable seed-to-soil contact.

Additionally, as shown in FIG. 2, the row unit 18 may include one or more seed hoppers 42, 44 and a fluid tank 46 supported on the row unit frame 34. In general, the seed hopper(s) 42, 44 may be configured to store seeds received from the seed tanks 20, which are to be deposited within the furrow as the row unit 18 travels across the field. For instance, in several embodiments, the row unit 18 may include a first seed hopper 42 configured to store seeds of a first seed type and a second hopper 44 configured to store seeds of a second seed type. However, both seed hoppers 42, 44 may be configured to store the same type of seeds. Furthermore, the fluid tank 46 may be configured to store fluid received from the fluid tank 22 (FIG. 1), which is to be sprayed onto the seeds dispensed from the seed hoppers 42, 44. For example, a sprayer assembly 48 mounted on the row unit frame 34 may be configured to spray the fluid stored in the fluid tank 22 onto the seeds.

Moreover, the row unit 18 may include a seed meter 50 supported on the row unit frame 34. In general, the seed meter 50 is configured to uniformly release seeds received from the seed hopper(s) 42, 44 for deposition within the furrow. For instance, in one embodiment, the seed meter 50 may be coupled to a suitable vacuum source (e.g., a blower powered by a motor and associated tubing or hoses) configured to generate a vacuum or negative pressure that attaches the seeds to a rotating seed disk of the seed meter 50, which controls the rate at which the seeds are output from the seed meter 50 to an associated seed tube 52. As shown in FIG. 2, the seed tube 52 may extend vertically from the seed meter 50 toward the ground to facilitate delivery of the seeds discharged from the seed meter 50 to the furrow.

The configuration of the row unit 18 described above and shown in FIG. 2 is provided only to place the present subject matter in an exemplary field of use. Thus, the present subject matter may be readily adaptable to any manner of seed planting unit configuration.

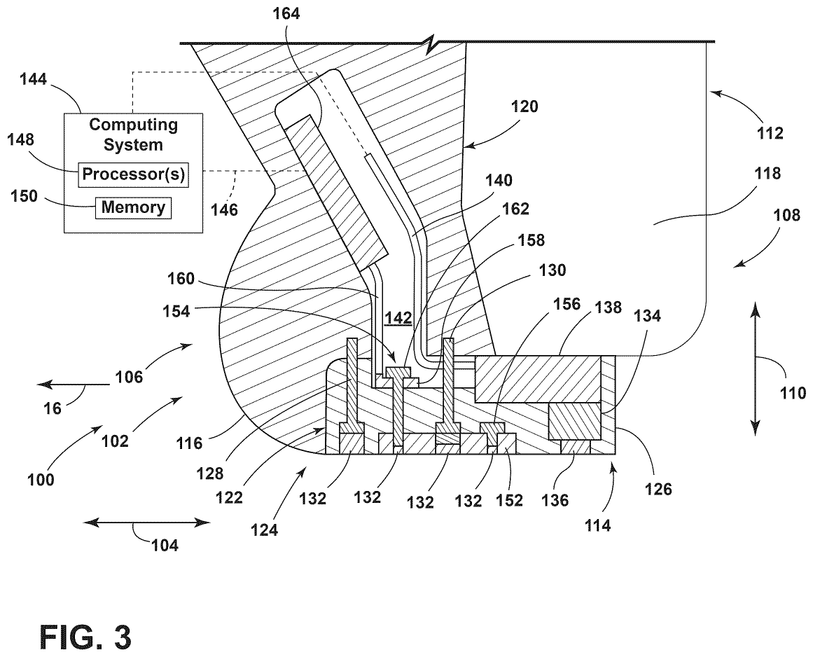

FIG. 3 illustrates a cross-sectional view of one embodiment of an optical sensor assembly 100 for a seed-planting implement. In general, the optical sensor assembly 100 will be described herein with reference to the seed-planting implement 10 and the row unit 18 described above with reference to FIGS. 1 and 2. However, the disclosed optical sensor assembly 100 can generally be utilized with seed-planting implements having any other suitable implement configuration and/or row units having any other suitable row unit configuration.

As shown in FIG. 3, the optical sensor assembly 100 includes the furrow firmer 102 of the row unit 18. More specifically, the furrow firmer 102 extends in a longitudinal direction 104 from a forward end 106 to an aft end 108, with the longitudinal direction 104 extending generally parallel to the direction of travel 16. Furthermore, the furrow firmer 102 extends in a vertical direction 110 from a top end 112 to a bottom end 114, with the vertical direction 110 extending generally perpendicular to the longitudinal direction 104. In some embodiments, the furrow firmer 102 includes a body 116 (e.g., a metallic casting) and a sleeve 118 (e.g., formed of sheet metal) coupled to an aft end 120 of the body 116. Additionally, the furrow firmer 102 defines a cavity 122 at the bottom end 114. For example, in the illustrated embodiment, the cavity 122 is defined at a bottom end 124 of the body 116. As will be described below, additional components of the optical sensor assembly 100 are positioned within the cavity 122, thereby allowing for the determination of the certain properties of the soil (e.g., its organic matter content) while not negatively impacting the furrow closing operation being performed by the closing disk(s) 40 (FIG. 2).

Furthermore, the optical sensor assembly 100 includes a non-electrically conductive housing 126. As shown, the non-electrically conductive housing 126 is positioned within the cavity 122. In general, the non-electrically conductive housing 126 is formed out of any suitable non-electrically conductive or otherwise electrically insulative material. For example, in some embodiments, the non-electrically conductive housing 126 may be formed of a polymeric material.

Additionally, the non-electrically conductive housing 126 may be mechanically coupled to the furrow firmer 102 in any suitable manner. More specifically, in some embodiments, the non-electrically conductive housing 126 may be mechanically coupled to the furrow firmer 102 via one or more fasteners. For example, in the illustrated embodiment, the non-electrically conductive housing 126 is mechanically coupled to the furrow firmer 102 (e.g., the body 116 of the furrow firmer 102) via a first fastener 128 and a second fastener 130. In some embodiments, caps 132 may be placed in the holes in which the first and second fasteners 128, 130 are received to prevent soil accumulation therein.

Moreover, the optical sensor assembly 100 includes an optical sensor 134 positioned within the cavity 122 and coupled to the non-electrically conductive housing 126. In general, the optical sensor 134 is configured to generate data indicative of one or more parameters or characteristics of the soil within the field. In some embodiments, the optical sensor 134 is configured to generate data indicative of the organic matter content of the soil. Thus, as will be described below, the data generated by the optical sensor 134 can be used to determine the organic matter content of the soil.

The optical sensor 134 may correspond to any suitable type of sensor configured to capture optical or light-based data or images. For example, in some embodiments, the optical sensor 134 may include one or more light-emitting sources (e.g., one or more LEDs) configured to emit light directed at the soil forming the bottom surface of the furrow. At least a portion of this emitted light may be reflected by the soil. Thus, the optical sensor 134 may also include one or more photodiodes configured to convert the reflected light into an electric current. Based on one or more parameters of this electric current (e.g., its voltage), the organic matter content or other characteristics of the soil can be determined.

In addition, the optical sensor assembly 100 includes a lens 136. In general, the lens 136 is the medium through which the optical sensor 134 views the soil forming or otherwise defining the furrow. As such, the lens 136 may focus and/or direct the light being emitted by the optical sensor 134 to a particular location on the bottom surface of the furrow. Similarly, the lens 136 may focus and/or direct the light being reflected by the soil to the optical sensor 134 (e.g., to its photodiode(s)).

In general, the optical sensor 134 and the lens 136 are oriented such that the optical sensor 134 has a field of view directed downward in the vertical direction 110 toward the bottom surface of the furrow. Thus, the lens 136 is generally positioned below the optical sensor 134 in the vertical direction 110. Moreover, the lens may generally be aligned with the optical sensor 134 in the longitudinal direction 104 (and/or in a lateral direction (not shown) that is perpendicular to the longitudinal direction 104 and the vertical direction 110).

Such positioning of the optical sensor 134 and the lens 136 within the furrow firmer 102 (e.g., at the bottom end 124 of the body 116 of furrow firmer 102) improves the operation of the seed-planting implement 10. More specifically, this positioning allows the optical sensor 134 to generate such data indicative of the organic matter content of the soil forming the bottom surface of the furrow while facilitating improved furrow closing operation over conventional optical sensors that bolt onto the seed-planting implement behind the furrow firmer.

In several embodiments, the lens 136 is coupled to the non-electrically conductive housing. For example, in some embodiments, the lens 136 may be adhesively coupled (e.g., via a suitable epoxy) to the non-electrically conductive housing 126. Such coupling prevents the lens 136 from rotating, twisting, or otherwise moving relative to the optical sensor 134 during operation as such movement could affect the emitted or reflected light in a manner resulting in inaccurate or inconsistent determinations of the organic matter content.

Furthermore, the optical sensor assembly 100 includes a circuit board 138. Specifically, in several embodiments, the circuit board 138 is electrically coupled to the optical sensor 134, such as via a suitable soldered connection. As shown, the circuit board 138 may be positioned above the optical sensor 134 in the vertical direction 110. Moreover, the circuit board 138 may be at least partially positioned between the non-electrically conductive housing 126 and the furrow firmer 102 in the vertical direction 110. Thus, the optical sensor 134 may be at least partially positioned between the circuit board 138 and the lens 136 in the vertical direction 110.

As mentioned above, the optical sensor 134 may be coupled to the non-electrically conductive housing 126. For example, in one embodiment, the optical sensor 134 may be coupled to a circuit board 138, which, in turn, is coupled (e.g., potted) to the non-electrically conductive housing 126.

Additionally, the optical sensor assembly 100 includes a wire 140 electrically coupled to the circuit board 138. More specifically, as shown in FIG. 3, the furrow firmer 102 and/or the non-electrically conductive housing 126 may define a passage 142. In this respect, the wire 140 may be routed at least partially through the passage 142. For example, one end of the wire 140 may be electrically coupled to the circuit board 138 (e.g., via a suitable soldered connection), while the opposing end of the wire 140 may be electrically coupled (e.g., directly or indirectly) to a computing system 144 (e.g., as indicated by a communicative link 146).

Moreover, as indicated above, the optical sensor assembly 100 includes the computing system 144 communicatively coupled to one or more components of the optical sensor assembly 100, the row unit 18, and/or the seed-planting implement 10. For instance, in some embodiments, the computing system 144 may be communicatively coupled to the circuit board 138 via the wire 140 and/or other components of the communicative link 146. Alternatively, the circuit board 138 may be part of the computing system 144. As such, the computing system 144 may be configured to receive data from the optical sensor 134, such as via the circuit board 138, the wire 140, and/or the communicative link 146. Such data may generally be indicative of the organic matter content of the soil within the field. In addition, the computing system 144 may be communicatively coupled to any other suitable components of the optical sensor assembly 100, the row unit 18, and/or the seed-planting implement 10, such as any other sensor(s) positioned within the cavity 122.

In general, the computing system 144 may include one or more processor-based devices, such as a given controller or computing device or any suitable combination of controllers or computing devices. Thus, in several embodiments, the computing system 144 may include one or more processor(s) 148 and associated memory device(s) 150 configured to perform a variety of computer-implemented functions. As used herein, the term “processor” refers not only to integrated circuits referred to in the art as being included in a computer, but also refers to a controller, a microcontroller, a microcomputer, a programmable logic circuit (PLC), an application specific integrated circuit, and other programmable circuits. Additionally, the memory device(s) 150 of the computing system 144 may generally comprise memory element(s) including, but not limited to, a computer-readable medium (e.g., random access memory RAM)), a computer-readable non-volatile medium (e.g., a flash memory), a floppy disk, a compact disk-read only memory (CD-ROM), a magneto-optical disk (MOD), a digital versatile disk (DVD) and/or other suitable memory elements. Such memory device(s) 150 may generally be configured to store suitable computer-readable instructions that, when implemented by the processor(s) 148, configure the computing system 144 to perform various computer-implemented functions. In addition, the computing system 144 may also include various other suitable components, such as a communications circuit or module, one or more input/output channels, a data/control bus, and/or the like.

The various functions of the computing system 144 may be performed by a single processor-based device or may be distributed across any number of processor-based devices, in which instance such devices may be considered to form part of the computing system 144. For instance, the functions of the computing system 144 may be distributed across multiple application-specific controllers or computing devices (e.g., the circuit board 138 may be part of the computing system 144).

In several embodiments, the computing system 144 may be configured to determine the organic material content of the soil based on data generated by the optical sensor 134. More specifically, as indicated above, the data generated by the optical sensor 134 is indicative of one or more characteristics of the soil, such as its organic matter content. Furthermore, as mentioned above, the computing system 144 is electrically or otherwise communicatively coupled to the optical sensor 134. In this respect, the computing system 144 may receive data from the optical sensor 134 during operation of the seed-planting implement 10. Based on this received data, the organic matter content of the soil can be determined. For example, the computing system 144 may include a look-up stored within its memory device(s) 150 correlating the optical sensor data with an organic matter content value for the soil.

Furthermore, as indicated above, other sensors may be positioned within the cavity 122 defined by the furrow firmer 102. Specifically, in several embodiments, an electrode 152 may be positioned within the cavity 122 for use in determining the electrical conductivity of the soil. In this respect, the electrode 152 may formed of a metallic material. For example, in the illustrated embodiment, the electrode 152 is configured as a metallic strip. In this respect, the non-electrically conductive housing 126 electrically isolates the electrode 152 from the furrow firmer 102. Moreover, the electrode 152 may be mechanically coupled to the non-electrically conductive housing 126 via one or more fasteners, such as a third fastener 154 and a fourth fastener 156. In some embodiments, one of the fasteners may be used to transmit electric current to and/or from the electrode 152. For example, in the illustrated embodiment, a terminal 158 coupled to one end of a wire 160 may be positioned between a head 162 of the third fastener 154 and the non-electrically conductive housing 126. The opposing end of the wire 160 may be coupled to a circuit board 164 positioned within an upper portion of the passage 142. The circuit board 164 may, in turn, be part of the computing system 144 or communicatively coupled to the computing system 144 (e.g., via the communicative link 146). In this respect, the computing system 144 may be configured to use the electrode 152 in combination with electrodes on other row units (e.g., one electrode of four row units) to determine the electrical conductivity of the soil.

This written description uses examples to disclose the technology, including the best mode, and also to enable any person skilled in the art to practice the technology, including making and using any devices or systems and performing any incorporated methods. The patentable scope of the technology is defined by the claims and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. An optical sensor assembly for a seed-planting implement, the optical sensor assembly comprising:
a furrow firmer configured to shape a furrow being formed in soil by the seed-planting implement, the furrow firmer extending in a vertical direction from a top end to a bottom end, the furrow firmer defining a cavity at the bottom end;
a non-electrically conductive housing positioned within the cavity;
an optical sensor positioned within the cavity and coupled to the non-electrically conductive housing; and
a lens through which the optical sensor views the soil defining the furrow.

2. The optical sensor assembly of claim 1, wherein the optical sensor and the lens are oriented such that the optical sensor has a field of view directed downward in the vertical direction toward a bottom surface of the furrow.

3. The optical sensor assembly of claim 1, wherein the lens is coupled to the non-electrically conductive housing.

4. The optical sensor assembly of claim 1, further comprising:
a circuit board electrically coupled to the optical sensor, the circuit board at least partially positioned between the non-electrically conductive housing and the furrow firmer in the vertical direction.

5. The optical sensor assembly of claim 4, wherein the optical sensor is at least partially positioned between the circuit board and the lens in the vertical direction.

6. The optical sensor assembly of claim 5, wherein at least one of the furrow firmer or the non-electrically conductive housing further defines a passage, the assembly further comprising:
a wire extending through the passage and electrically coupled to the circuit board.

7. The optical sensor assembly of claim 6, further comprising:
a computing system electrically coupled to the optical sensor, the computing system configured to determine an organic material content of the soil based on data generated by the optical sensor.

8. The optical sensor assembly of claim 1, wherein the non-electrically conductive housing is mechanically coupled to the furrow firmer via one or more fasteners.

9. The optical sensor assembly of claim 1, wherein the non-electrically conductive housing is formed of a polymeric material.

10. A row unit for a seed-planting implement, the row unit comprising:
a row unit frame;
a disk opener rotatably coupled to the row unit frame, the disk opener configured to form a furrow within soil of a field as the seed-planting implement travels across the field;
a furrow firmer coupled to the row unit frame, the furrow firmer configured to shape the furrow, the furrow firmer extending in a vertical direction from a top end to a bottom end, the furrow firmer defining a cavity at the bottom end;
a non-electrically conductive housing positioned within the cavity;
an optical sensor positioned within the cavity and coupled to the non-electrically conductive housing; and
a lens through which the optical sensor views the soil defining the furrow.

11. The row unit of claim 10, wherein the optical sensor and the lens are oriented such that the optical sensor has a field of view directed downward in the vertical direction toward a bottom surface of the furrow.

12. The row unit of claim 10, wherein the lens is coupled to the non-electrically conductive housing.

13. The row unit of claim 10, further comprising:
a circuit board electrically coupled to the optical sensor, the circuit board at least partially positioned between the non-electrically conductive housing and the furrow firmer in the vertical direction.

14. The row unit of claim 13, wherein the optical sensor is at least partially positioned between the circuit board and the lens in the vertical direction.

15. The row unit of claim 14, wherein at least one of the furrow firmer or the non-electrically conductive housing further defines a passage, the assembly further comprising:
a wire extending through the passage and electrically coupled to the circuit board.

16. The row unit of claim 10, further comprising:
an electrode positioned within the cavity for use in determining an electrical conductivity of the soil.

17. The row unit of claim 16, wherein the non-electrically conductive housing electrically isolates the electrode from the furrow firmer.

18. The assembly of claim 17, wherein the non-electrically conductive housing is formed of a polymeric material and the electrode is formed of a metallic material.

19. A seed-planting implement, comprising:
a toolbar; and
a plurality of row units supported on the toolbar, at least one row unit of the plurality of row units comprising:
a furrow firmer configured to shape a furrow being formed by the row unit, the furrow firmer extending in a vertical direction from a top end to a bottom end, the furrow firmer defining a cavity at the bottom end;
a non-electrically conductive housing positioned within the cavity;
an optical sensor positioned within the cavity and coupled to the non-electrically conductive housing; and
a lens through which the optical sensor views the soil defining the furrow.

20. The seed-planting implement of claim 19, wherein the optical sensor and the lens are oriented such that the optical sensor has a field of view directed downward in the vertical direction toward a bottom surface of the furrow.
